(19) United States
(12) Patent Application Publication
(10) Pub. No.: US 2022/0354990 A1
(43) Pub. Date: Nov. 10, 2022

(54) METHOD FOR BIOMATERIAL FUNCTIONALIZATION WITH IMMOBILIZED EXTRACELLULAR VESICLES

(71) Applicant: Carnegie Mellon University, Pittsburgh, PA (US)

(72) Inventors: Xi Ren, Pittsburgh, PA (US); Phil G. Campbell, Pittsburgh, PA (US); Yunhui Xing, Pittsburgh, PA (US); Saigopalakrishna Saileelaprasa Yerneni, Pittsburgh, PA (US)

(73) Assignee: Carnegie Mellon University, Pittsburgh, PA (US)

(21) Appl. No.: 17/741,271

(22) Filed: May 10, 2022

Related U.S. Application Data
(60) Provisional application No. 63/186,470, filed on May 10, 2021.

(51) Int. Cl. A61L 27/36

(52) U.S. Cl. CPC A61L 27/3633; A61L 27/3687; A61L 2430/40

(57) ABSTRACT

A method of immobilizing extracellular vesicles in an extracellular matrix material provides for improved extracellular vesicle retention in vitro and in vivo. Extracellular matrix materials, such as collagen, are functionalized with chemicals that are complimentary to ligands disposed on the surface of the extracellular vesicle. In one example embodiment, collagen is functionalized with dibenzocyclooctyne and the extracellular vesicle is functionalized with an azide tag. The extracellular vesicle is immobilized within the collagen through a click reaction involving the dibenzocyclooctyne and the azide tag.

### CROSS-REFERENCE TO RELATED APPLICATIONS

This application claims the benefit under 35 U.S.C. § 119 of U.S. Provisional Application Ser. No. 63/186,470, filed May 10, 2021, which is incorporated herein by reference.

### STATEMENT REGARDING FEDERALLY SPONSORED RESEARCH

Not applicable.

### BACKGROUND OF THE INVENTION

The present disclosure generally relates to tissue engineering with extracellular vesicles (EVs). More specifically, the disclosure relates to a method of immobilizing EVs within an extracellular matrix, such as collagen, to increase the retention and bioactivity of the EVs in tissue engineering and regenerative medicine applications.

Mesenchymal stem cells (MSCs) can be obtained from a wide variety of tissues and have demonstrated robust efficacy in many applications. Accumulating evidence suggests that the therapeutic benefits of MSCs are to a major extent carried out through paracrine mechanisms via the MSC secretome, and in particular in the form of EVs carrying cargoes such as nucleotides, lipids, enzymes, signal transduction proteins, immunomodulatory cytokines, and growth factors. The therapeutic efficacy of MSC-derived EVs has been well documented in a variety of injury repair models, through mechanisms including but not limited to inducing cellular proliferation and migration, angiogenesis, and anti-apoptotic and anti-inflammatory effects. Furthermore, compared to therapies using living cells, EV-based therapeutics are less likely to trigger adverse immune responses, are exempt from uncontrolled growth of foreign cells, and do not suffer from the logistics and regulatory concerns of therapies based on living cells.

Nanoscale EVs represent a unique cellular derivative that reflect the therapeutic potential of MSCs toward tissue engineering. However, systemically administered EVs undergo rapid clearance and typically are without a focused targeted delivery, thus reducing their effectiveness in therapeutic regenerative therapies. Therefore, it would be advantageous to develop a method of immobilizing EVs to enable controlled delivery and prolonged retention of EVs, making possible the long-term support of tissue regeneration and repair among other therapeutic applications.

### BRIEF SUMMARY

According to embodiments of the present disclosure is a method of immobilizing EVs in a matrix material, such as collagen. In one embodiment, the method utilizes chemoselective immobilization of MSC-derived EVs, bearing metabolically incorporated azide ligands, within a dibenzocyclooctyne-modified collagen hydrogel, enabling long-term EV spatial retention. Azide-EVs exhibit comparable morphological and functional properties as their non-labeled EV counterparts and, when immobilized within collagen hydrogel implants, they elicited more robust host cell infiltration, angiogenic and immunoregulatory responses including vascular ingrowth and macrophage recruitment compared to what can be achieved using ten times the higher dose of non-immobilized EVs. The immobilized EVs can enable a wide range of applications to spatially promote vascularization and host integration relevant to tissue engineering and regenerative medicine applications.

### BRIEF DESCRIPTION OF THE SEVERAL VIEWS OF THE DRAWINGS

FIG. 1 is a flowchart showing the process of EV immobilization, according to one embodiment.

FIG. 2 is a graph showing EV labeling.

FIG. 3 is a graph showing EV immobilization for various materials.

FIG. 4 is a flowchart showing functionalization of an extracellular matrix material.

FIG. 5 is a graph showing functionalized collagen versus control collagen.

FIG. 6 is a graph showing EV retention as a function of time.

FIG. 7 is a graph depicting cell concentration.

FIG. 8 is a graph depicting hemoglobin concentration.

FIG. 9 is a graph depicting cell concentration.

FIG. 10 is a graph depicting fluorescent intensity.

FIG. 11 is a graph depicting fluorescent intensity.

FIG. 12 is a graph depicting fluorescent intensity.

### DETAILED DESCRIPTION

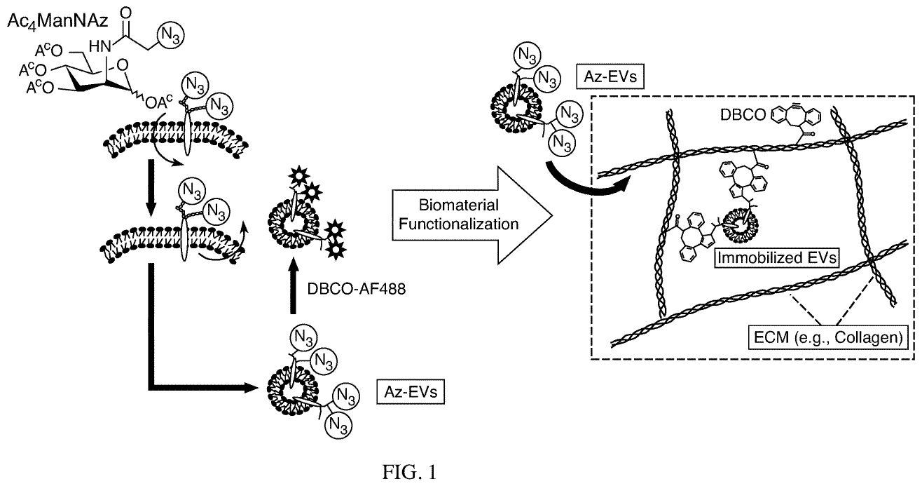

According to embodiments of the disclosure is a method of creating immobilized extracellular vesicles (EV) by combining metabolically labeled EVs and click-reactive extracellular matrix materials. In one example embodiment, azide-labeled mesenchymal stem cell-derived EVs (MSC-EVs) are formed using metabolic glycan engineering and are combined with dibenzocyclooctyne (DBCO)-labeled collagen, which is formed using amine reactive chemistry. More specifically, the method utilizes an azide-to-DBCO click reaction to immobilize Azide-EVs within biomaterials (such as collagen hydrogels) bearing complementary DBCO ligands. FIG. 1 is a schematic showing the method, according to one embodiment, for creating EVs bearing surface azide decoration, which allows the EVs to be immobilized in DBCO-labeled collagen.

Azide-to-alkyne click chemistry allows chemoselective conjugation between bioinert ligands, permitting the biomaterial-anchored delivery of bioactive molecules. Click-reactive chemical ligands can be incorporated into living cells and their derivatives via metabolic protein engineering through either translational incorporation of unnatural amino acids or post-translational incorporation of glycan probes. As a cellular derivative, EVs can be labeled with azide ligands through metabolic glycan engineering using azido-monosaccharide probes, such as N-azidoacetylmannosamine-tetraacylated (Ac₄ManNAz), which can also allow in vivo tracking and imaging of the labeled EVs.

Metabolic glycan engineering using azide or alkyne-modified monosaccharide probes has demonstrated robust efficacy in labeling glycoproteins that are most commonly found on the exterior surface of the plasma membrane as well as in the extracellular matrix (ECM). As membrane encapsulated vesicular derivatives of living cells, the surface of EVs is also decorated with extensive glycan groups associated with glycolipid and glycoproteins. The method described herein can be used for biomaterial functionalization using the metabolically incorporated azide ligands on the EV surface as chemical handles for EV immobilization within biomaterials for tissue engineering applications.

The method represents a platform technology as EVs are the universal communicator across all living organisms. Moreover, azide-to-DBCO click conjugation can be carried out in most biological systems and requires minimal optimization due to their high reactivity towards each other and inertness to most native biological molecules. The method can be used for engineering EVs derived from most cell sources, where membrane protein glycosylation is a universal phenomenon. Furthermore, the azide incorporation process is carried out using physiologic post-translational modification of newly synthesized proteins, and azide labeling possesses minimal interference to the normal physiological processes of cells and animals. Thus, the surface-engineered EVs retain their inherent functions.

As shown in FIG. 1, azide labeling of mesenchymal stem cell (MSCs)-derived EVs can be performed using the inherent cellular metabolic pathway for sialic acid glycosylation. In this process, MSCs are exposed to the azide-monosaccharide probe (Ac₄ManNAz, 50 μM). The resulting MSC-conditioned medium is then collected and processed for EV isolation, before additional steps in the method.

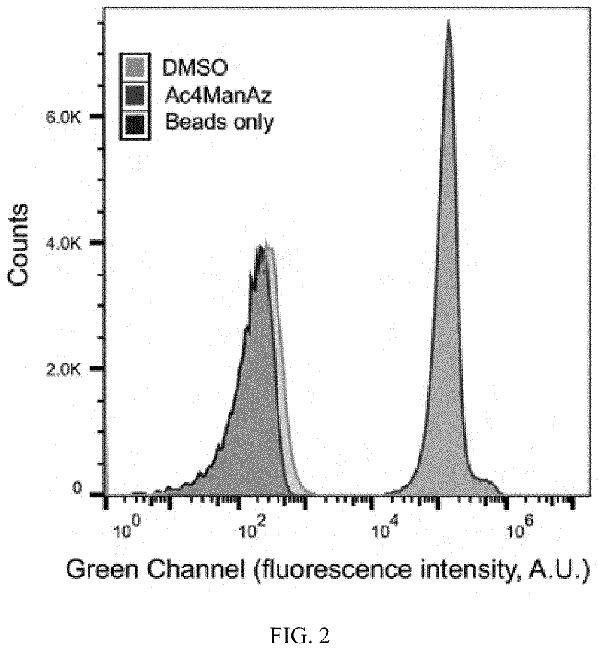

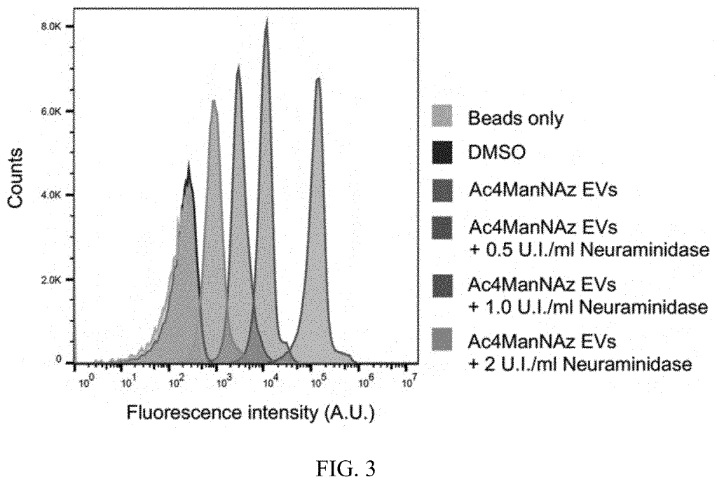

To confirm azide-tags on MSC-EVs, click conjugation can be performed with a fluorescent probe, such as DBCO-Alexa Fluor 488 (AF488), and analyzed by on-bead flow cytometry, which demonstrates robust azide labeling specifically on MSC-EVs derived from cells pretreated with Ac₄ManNAz. To validate the incorporation of the azido-monosaccharide probe as a replacement of native sialic acid, Az-EVs were conjugated with DBCO-AF488 and then pretreated with neuraminidase, which cleaves terminal sialic acid residues on glycans and analyzed by on-bead flow cytometry. FIG. 2 is a graph showing effective azide labeling of EVs derived from MSCs pretreated with Ac₄ManNAz, while the azide labeling in control EVs derived from MSCs pretreated with DMSO (the solvent) was almost undetectable. Further, FIG. 3 shows a neuraminidase-dose-dependent reduction of AF488 fluorescence intensity on Azide-EVs, confirming sialic acid labeling.

To perform the analysis depicted in FIGS. 2-3, CD63-conjugated magnetic beads are prepared according to the following process. First, monoclonal anti-CD63 antibody is biotinylated using a one-step antibody biotinylation kit according to manufacturer's instructions. Biotinylated CD63 antibody (5 μg) is then incubated with thoroughly washed 0.5 mL of streptavidin-coated magnetic beads (1×10⁸ beads/mL) for 1 hour at 25° C. under constant agitation. EVs are incubated with 100 μL of CD63-conjugated magnetic beads for 16 hours at 4° C. under constant agitation, washed three times with phosphate-buffered saline (PBS), and analyzed on an Accuri C6 flow cytometer connected to an Intellicyt HyperCyt autosampler at 20,000 events/treatment. For neuraminidase treatment groups, the isolated EVs are pre-treated with 0.5, 1 or 2 IU/mL neuraminidase at 37° C. for 30 min followed by EV purification over Sepharose 2B column before binding to magnetic beads.

The membrane surface charge of EVs and target cells play an essential role in regulating EV internalization. The metabolic azide labeling targets the terminal sialic acids on EV surface proteins, which are negatively charged and play essential functions in mediating receptor-ligand signaling. Accordingly, when the sialic acids are removed from the EV surface using neuraminidase, an obvious decrease in the cellular uptake of EVs is observed. This further confirms the critical function of sialic acid in regulating EV internalization and that the sialic acid-tagged metabolic azide labeling procedure does not interfere with the physiological functions of EVs.

Referring again to FIG. 1, collagen is depicted as the ECM in this example embodiment. Native ECM materials possess desired biochemical and biophysical cues to guide communication, cellular engraftment, remodeling, and formation of new tissues, and act as a vehicle for EV delivery both naturally and via engineered constructs. Collagen is a highly abundant ECM component found in most mammalian tissues and is accordingly one of the most widely used biomaterial building blocks in tissue engineering. Collagen can be fabricated into a broad variety of shapes and sizes and is compatible with the latest advances in tissue fabrication technologies. While collagen is shown as the extracellular matrix material in this example, other matrix materials and biomaterials may be used, such as fibrin, gelatin, fibronectin, Matrigel, elastin, and decellularized ECM. The matrix material may also include other natural and synthetic biomaterials that can be conjugated with an azide-reactive cyclooctyne or phosphine group.

The ECM is an acellular three-dimensional structure present in almost all tissues and organs. Besides serving as structural support for cell engraftment, it is also a rich signaling source for regulating cellular activities through the diverse range of biomolecular species embedded within the ECM, such as growth factors and EVs. Thus, using collagen alone is usually insufficient for mimicking the complex microenvironment that is necessary to trigger the desired cellular responses during tissue regeneration. EVs delivered by hydrogel without immobilization usually results in a rapid burst release and swift clearance of EVs, which lead to poor therapeutic outcomes. As a result, the present method modifies the ECM with DBCO or other cyclooctyne reagents, enabling immobilization of the EVs within the ECM. Other cyclooctyne reagents may include difluorinated cyclooctyne (DIFO), biarylazacyclooctynone (BARAC), and bicyclononyne (BCN). Alternatively, the ECM may be functionalized with phosphine to enable phosphine-to-azide Staudinger ligation reactions.

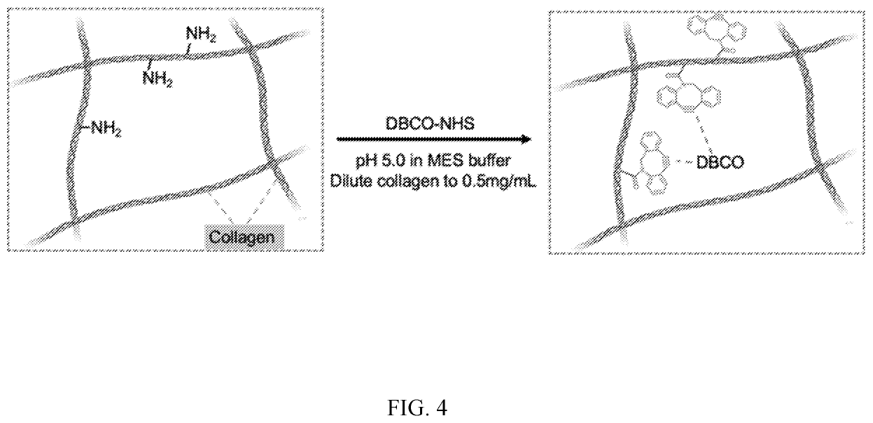

Using the amine-reactive carbodiimide chemistry, DBCO-conjugated, clickable collagen (DBCO-collagen) is engineered for subsequent chemoselective immobilization of Azide-EVs. To enable effective DBCO-N-Hydroxysuccinimide (DBCO-NHS) conjugation to the primary amines of collagen (such as type-I collagen) while maintaining collagen solubility (preventing polymerization), the conjugation reaction is performed with diluted collagen (0.5 mg/mL) at pH 5.0 in MES buffer (see FIG. 4). Besides amine-reactive carbodiimide chemistry, other reaction mechanisms, such as carboxyl/carbonyl reactive chemistry and sulfhydryl reactive chemistry for example, can also be used based on the biochemical nature of the materials. Similar approaches can be applied to other ECM materials and to other cyclooctyne reagents and phosphine to generate azide-reactive ECM materials.

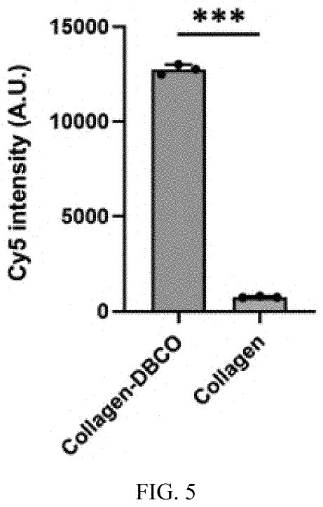

To confirm functionalization with DBCO, the resulting DBCO-collagen can be incubated with fluorescent Azide-Cy5 for potential azide-to-DBCO conjugation to take place, which is analyzed using dot blot analysis and fluorescence quantification. Robust Cy5 fluorescence can be observed in DBCO-collagen dot blot while an unmodified collagen control shows undetectable levels under the same condition of equal protein loading (see FIG. 5), demonstrating effective covalent modification of collagen with DBCO.

By way of further detail of the method depicted in FIG. 1, the following process steps can be performed in one example embodiment. First, mesenchymal stem cells (MSCs) are extracted and maintained in a culture medium. Alternatively, MSCs maintained in a growth medium can be obtained from a commercial source. In yet another alternative embodiment, EVs can be obtained from any cell source where the EVs are biologically active. The MSCs are then transferred to a growth medium containing 50 μM Ac₄ManNAz or similar click-reactive, metabolic labeling probe. For example, N-azidoacetylgalactosamine-tetraacylated (Ac₄GalNAz) and N-azidoacetylglucosamine-tetraacylated (Ac₄GlcNAz) are other azides that can be used to target glycosylation. Azide probes targeting amino acids, such as L-Azidohomoalanine (AHA), can also be used. Cells are then cultured for 72 hours before medium collection. The azide probes are introduced to the cell culture to label the EVs with azide. For example, azide probes (e.g. monosaccharides or amino acids decorated with an azide group) are added to the cell culture, where the cells metabolize the probes to present the azide group from these probes to EV's surface.

Next, EVs from the resulting conditioned medium are isolated by size exclusion chromatography (SEC). For example, conditioned medium can be centrifuged at 2,000×g for 10 min at 4° C. and then at 10,000-14,000×g for 30 min at 4° C. The supernatant is passed through a 0.22 μm-pore Millipore filter and EVs are isolated by mini-SEC using 1.5 cm×12 cm mini-columns packed with 10 mL of a filtration base matrix equilibrated with phosphate-buffered saline (PBS). The supernatant (1.0 mL) is loaded onto the column and five 1 mL fractions corresponding to the void volume peak are collected by running PBS over the column. Isolated EVs can either be used immediately (within 24 hours), placed at −80° C. for long-term storage, or lyophilized.

In parallel to labeling the EVs with a click-reactive ligand and isolating them, DBCO is conjugated onto collagen to form DBCO-collagen. In this example, bovine type I collagen is diluted in 100 mM MES buffer (pH 5) at 0.5 mg/mL and is then incubated with 200 μM DBCO-NHS ester with 10% DMSO at room temperature (RT) for 3 hours. Following conjugation, the reaction mixture is extensively dialyzed with 10,000-dalton molecular-weight-cutoff (MWCO) ultrafiltration filters using 0.1 M acetic acid solution to remove unconjugated DBCO molecules and exchange buffer. Finally, the Azide-EVs (Az-EVs) and DBCO-collagen are combined through a click reaction.

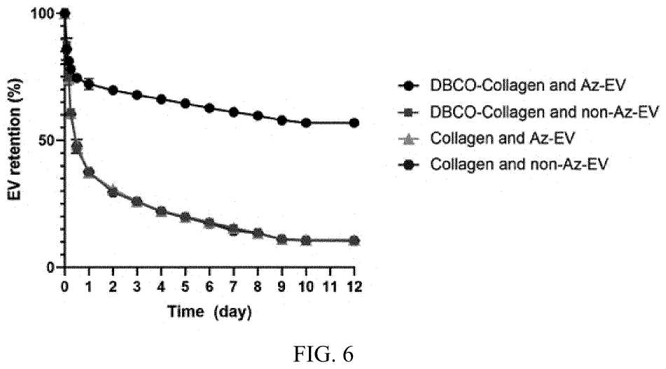

FIG. 6 is a graph that depicts the long-term EV retention in collagen gels by comparing Az-EVs versus non-Az-EVs and comparing DBCO-collagen versus unmodified collagen utilizing ²⁵I-radiolabeled MSC-EVs. The combination of ¹²⁵I-Az-EVs and DBCO-collagen followed by overnight pre-incubation at 4° C. before gelation led to superior long-term EV retention (>60% after 12 days) compared to all other ¹²⁵I-EV-collagen combinations (<15% after 12 days) (see FIG. 6). The results also show enhanced Az-EV retention in DBCO-collagen gel without pre-incubation. Taken together, these results demonstrate that combination of clickable DBCO-collagen and Az-EVs enables EV-biomaterial retention with superior stability.

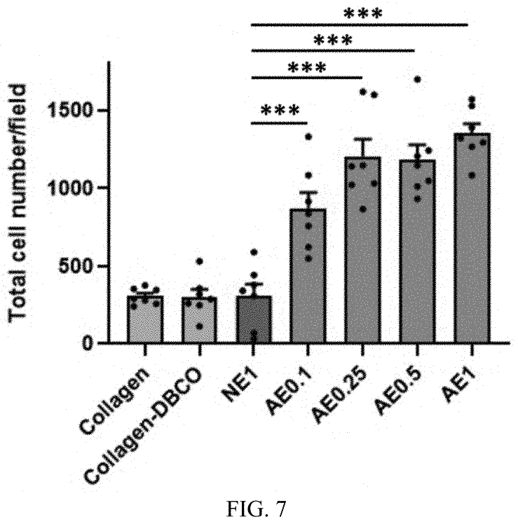

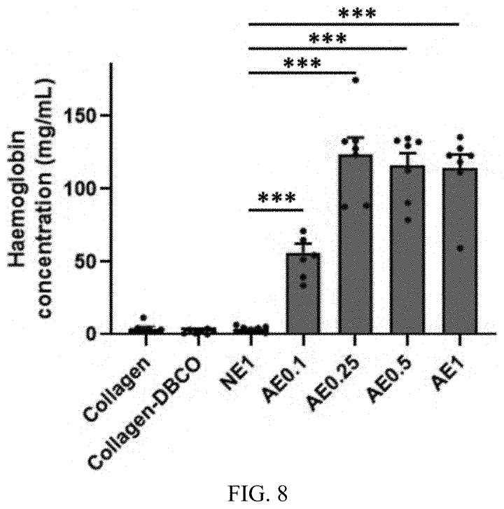

FIGS. 7-8 show an evaluation of in vivo angiogenic activities of EV-immobilized collagen gels. MSC-derived EVs are well known regarding their inherent pro-angiogenic functions. To investigate how enhanced. EV retention, achieved through combining DBCO-collagen and Az-EVs, regulates biornaterial vascularization in vivo, a subcutaneous plug model in mice was utilized. Varying doses of Az-EVs (0.1, 0.25, 0.5, and 1 μg per implant) were mixed with DBCO-collagen (3 mg/mL) prior to gelation and implantation into C57BL/6 mice, and the constructs were harvested for analysis 7 days post-implantation. Control implants consisted of DBCO-collagen gel plugs functionalized with non-Az-EVs, DBCO-collagen gel plugs without EVs, and unmodified collagen gel plugs without EVs. Robust host vascular ingrowth can be visually observed in DBCO-collagen gel plugs immobilized with as low as 0.1 μg Az-EVs, as compared to minimal angiogenic response in DBCO-collagen gel plugs with 10 times higher dose of non-Az-EVs (1 μg).

To comprehensively assess in vivo angiogenic responses, the harvested implants were divided into two halves, one half for host hemoglobin quantification and the other half for histological analysis. Based on H&E staining, enhanced cell penetration was observed across all in Az-EV groups (0.1, 0.25, 0.5 and 1 μg) compared to the non-Az-EV group at 1 μg dose (see FIG. 7, which shows quantification of total cell penetration). Furthermore, functionalization of Az-EVs dose-dependently enhanced host hemoglobin presence (indicative of host blood perfusion) in the implants, which plateaued at 0.25 μg Az-EVs per DBCO-collagen implant (see FIG. 8, which shows hemoglobin quantification in harvested implants).

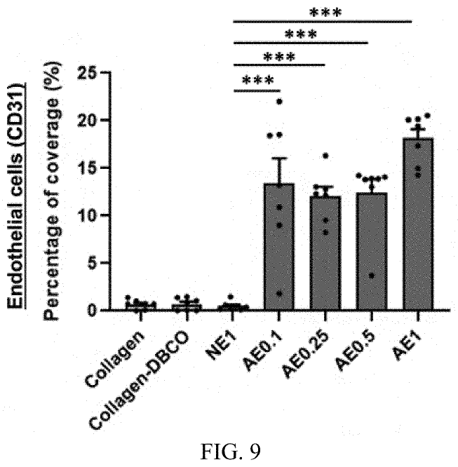

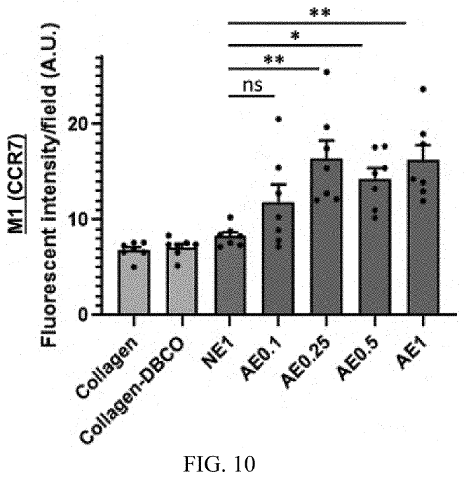

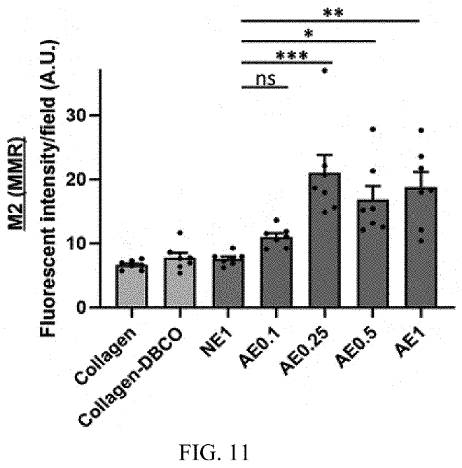

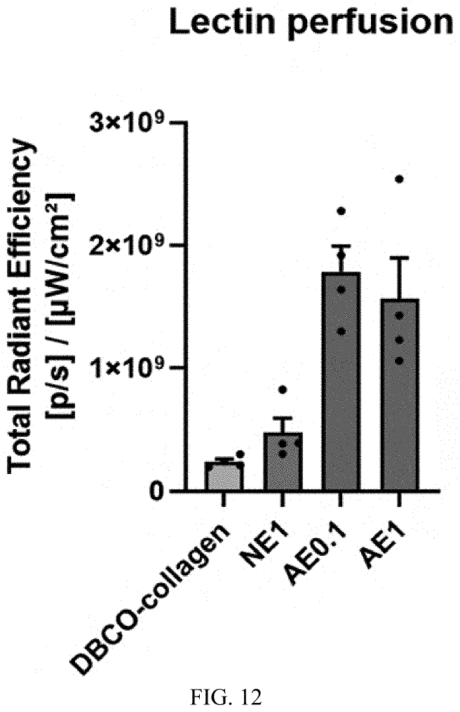

Robust induction of host vascular ingrowth is observed in all dosage group of Az-EVs in DBCO-collagen gel plugs, as indicated by the staining and quantification of host endothelial CD31 (see FIG. 9, which shows quantification of host vascular ingrowth via endothelial CD31 staining). Further, assessment of macrophage invasion was performed through the staining and quantification of CCR7 (M1 macrophages) and MMR (M2 macrophages). All Az-EVs in DBCO-collagen groups demonstrated in a significant increase in the infiltration of both M1 and M2 macrophages (see FIG. 10, which shows quantification of M1 macrophage staining (CCR7) and FIG. 11, which shows quantification of M2 macrophage staining (MMR)), which are well documented to promote vascularization. Functional angiogenic invasion into the implants was also observed, with the 0.1 μg Az-EVs groups showed significantly higher fluorescent intensity together with complete functional vascular network structure formation (see FIG. 12). A connection between the implants and the tissue in vicinity and the vascular formation was also present.

Vascularization is essential for successful implantation of bioengineered tissues. The implanted tissues rely on sufficient neovascularization to deliver oxygen and nutrients to support long-term tissue viability and functions. This is especially critical for thick or large-volume tissue scaffolds, where insufficient or delayed host vascular perfusion leads to tissue hypoxia, cell apoptosis, and impaired implant function. Thus, achieving effective and functional vascularization within the tissue engineered implants is of critical importance in their translation towards clinical therapies. The method described herein creates collagen functionalized through EV immobilization and shows robust ability to improve host vascularization of implanted hydrogels.

The chemoselective EV immobilization technique is applicable to other commonly used biomaterials, such as fibrin, elastin, and decellularized ECM, and is fully compatible with three-dimensional biofabrication techniques, such as bioprinting, for engineering complex tissues consisting of multiple materials and cells. For example, the method can be applied to a variety of tissue engineering and regenerative applications, such as promoting graft (cellular and acellular) vascularization, promoting post-ischemic injury repair, promoting wound healing, and promoting the regeneration of tissues (such as bone, lung, liver, muscle, skin, kidney, heart, blood vessel, pancreas, intestine, and stomach). In other applications, the method can be used in applications related to the modulation of host immune response to implanted materials/tissue constructs and transplanted organs, and toward the improvement of the viability and functional outcome of transplanted organs, including, liver, lung, pancreatic islet, muscle, skin, kidney, heart, blood vessel, intestine, stomach, and bone.

The EV-biomaterial functionalization platform is readily expandable to promote regeneration of other tissues, such as bone, muscle and skin. Beyond the EV cell source and its inherent therapeutic properties, EVs can be further engineered to deliver not only proteins but also nucleotides and drugs as additional exogenous EV cargos either on the surface or within the lumen of EVs. These techniques have the potential to enable long-term modulation of specific regenerative processes. In addition, due to the robust and localized angiogenic responses induced by the EV-collagen system, the method is a useful embedding and delivery vehicle for organotypic cells, organoids, and tissues.

When used in this specification and claims, the terms “comprises” and “comprising” and variations thereof mean that the specified features, steps, or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure. Although certain example embodiments of the invention have been described, the scope of the appended claims is not intended to be limited solely to these embodiments. The claims are to be construed literally, purposively, and/or to encompass equivalents.

What is claimed is:

1. A process for creating an immobilized extracellular vesicle comprising:
functionalizing an extracellular vesicle with an azide ligand;
functionalizing an extracellular matrix material with an azide-complimentary reagent;
immobilizing the extracellular vesicle within the extracellular matrix material through a click reaction between the azide ligand and the azide-complimentary ligand.

2. The process of claim 1,
wherein the azide-complimentary reagent comprises cyclooctyne; and
wherein the click reaction comprises an azide-to-cyclooctyne click reaction.

3. The process of claim 1,
wherein the azide-complimentary reagent comprises a phosphine reagent; and
wherein the reaction comprises an azide-to-phosphine Staudinger reaction.

4. The process of claim 1, wherein the extracellular matrix material comprises a natural or synthetic biomaterial capable of being conjugated with a cyclooctyne or phosphine ligand.

5. The process of claim 1, wherein the extracellular matrix material is selected from the group consisting of collagen, fibrin, gelatin, fibronectin, Matrigel, elastin, and other decellularized or extracted extracellular matrix.

6. The process of claim 2, wherein the cyclooctyne reagent is selected from the group consisting of dibenzocyclooctyne, difluorinated cyclooctyne, biarylazacyclooctynone, and bicyclononyne.

7. The process of claim 1, further comprising:
isolating the extracellular vesicle from a mesenchymal stem cell culture.

8. The process of claim 1, further comprising:
isolating the extracellular vesicle from a cultured cell source having biologically active extracellular vesicles.

9. The process of claim 1, where functionalizing an extracellular vesicle with an azide ligand comprises:
attaching the azide ligand through metabolic glycan or amino acid engineering.

10. The process of claim 9, wherein the metabolic glycan and amino acid engineering targets the extracellular vesicle's native pathway.

11. The process of claim 9, wherein the metabolic glycan engineering targets the extracellular vesicle's native pathway for sialic acid glycosylation or other glycosylation pathways and the metabolic amino acid engineering targets the native pathway for extracellular vesicle protein translation.

12. The process of claim 1, further comprising:
deriving the extracellular vesicle from a cell culture; and
attaching the azide ligand to the extracellular vesicle by introducing an azide probe into the cell culture.

13. The process of claim 12, wherein the azide probe is selected from the group consisting of N-azidoacetylmannosamine-tetraacylated, N-azidoacetylgalactosamine-tetraacylated, N-azidoacetylglucosamine-tetraacylated, and L-Azidohomoalanine.

14. The process of claim 1, where functionalizing an extracellular matrix material with the azide-complimentary reagent comprises:
attaching a cyclooctyne or phosphine reagent to extracellular matrix through amine-reactive chemistry, carboxyl/carbonyl reactive chemistry, or sulfhydryl reactive chemistry.

15. The process of claim 1, wherein the immobilized extracellular vesicle is used in an application selected from the group consisting of: graft vascularization, post-ischemic injury repair, wound healing, tissue regeneration, immune response modulation to implanted material and/or organ, and viability and functional improvement for transplanted organs.

16. The process of claim 15, wherein the tissue is selected from the group consisting of: bone, lung, liver, skin, kidney, heart, blood vessel, pancreas, intestine, and stomach.

17. The process of claim 15, wherein the organ is selected from the group consisting of: liver, lung, pancreas, muscle, skin, kidney, heart, blood vessel, intestine, stomach, and bone.

18. The process of claim 1, further comprising:
associating a cargo with the extracellular vesicle.

19. A biomaterial created by the process of claim 1.

20. A biomaterial comprising:
an extracellular vesicle functionalized with an azide; and
an extracellular matrix material functionalized with an azide-complimentary reagent, wherein the extracellular vesicle is attached to the extracellular matrix material via a conjugation between the azide and the azide-complimentary reagent.
